(19) United States
(12) Patent Application Publication
(10) Pub. No.: US 2019/0292510 A1
(43) Pub. Date: Sep. 26, 2019

(54) Dual-Purpose Viral Transduction and Electroporation Device

(71) Applicant: The Charles Stark Draper Laboratory, Inc., Cambridge, MA (US)

(72) Inventors: Vishal Tandon, Somerville, MA (US); Jonathan R. Coppeta, Windham, NH (US); Kenneth Kotz, Newton, MA (US); Heena K. Mutha, Cambridge, MA (US); Jenna Leigh Balestrini, Boston, MA (US)

(21) Appl. No.: 16/359,629

(22) Filed: Mar. 20, 2019

Related U.S. Application Data
(60) Provisional application No. 62/645,277, filed on Mar. 20, 2018.

(51) Int. Cl. C12M 1/42; C12N 13/00; B01L 3/00; C12M 1/34; C12N 15/87

(52) U.S. Cl. CPC C12M 35/02; C12N 13/00; C12N 15/87; C12M 41/00; B01L 3/5027

(57) ABSTRACT

A viral transduction and/or electroporation device has s a membrane separating two chambers and two electroporation electrodes for the chambers. An electrical voltage source is used for establishing an electrical field across the membrane and between the two electrodes. In operation, fluid is flowed into the chambers including fluid containing electroporation cargo and viral transduction solution and an electrical field is established across the membrane and between the electrodes to electroporate cells pinned to the membrane and transfecting the cells.

### RELATED APPLICATIONS

This application claims the benefit under 35 USC 119(e) of U.S. Provisional Application No. 62/645,277, filed on Mar. 20, 2018, which is incorporated herein by reference in its entirety.

### BACKGROUND OF THE INVENTION

U.S. Prov. Pat. Appl. Ser. No. 62/438,203, by Borenstein, et al., concerns a microfluidic electroporation device for end-to-end cell therapies. The device includes a top channel that contains cells and a bottom channel that contains cargo. They are separated by a membrane. It utilizes top and bottom manifolds for flow immobilization of the cells. These manifolds are used to distribute flow along the length of the channels such that the vertical flow rate along the length of the channels is uniform.

U.S. Pat. Appl. Pub. No. US 2017/1349912 A1, by Borenstein, et al., describes systems and methods for transduction, activation, and otherwise treating cells. In the systems, cells are introduced into an inner layer of a multi-layered stack that defines at least one flow chamber and cell entrainment regions. Vertical flow through the stack entrains the cells in the cell entrainment regions along with genetic information introduction agents or other additives. The cells are then washed using reverse vertical flow and collected from the device.

U.S. patent application Ser. No. 16/137,478, by Kotz, et al., filed Sep. 20, 2018, describes an apparatus for genetic modification of cells. It describes a transduction device that uses a semipermeable membrane having pores dimensioned to allow the passage of fluid and prevent the passage of cells and viral particles. The membrane is positioned between two plates. The plates define chambers with respect to the membrane and ports are provided in the plates in order to allow flow into and out of the chambers.

### SUMMARY OF THE INVENTION

Methods for genetic modification of cells typically fall into one of two categories: (1) viral transduction methods, and (2) non-viral transfection methods. Existing technology that is designed to enhance genetic modification ultimately relies on one of these two fundamental methods (typically electroporation for non-viral transfection), and therefore commits the user to either viral transduction or electroporation.

Existing solutions have not provided a straightforward way to perform both types of genetic modification in the same device.

According to embodiments of the present invention, viral transduction (along with preconditioning/activation of cells needed in some cases) and electroporation can be conducted in the same device, affording the user additional versatility. Practicing the invention can also enable performing sequential genetic modifications using both viral and non-viral vectors.

In some of its aspects, the invention can improve transfection rates by co-localization. Thus, the invention is unique over some previous solutions in that it can provide a means to co-localize cells and vector, a feature that is not typically available in commercial electroporation devices. For viral vectors, exiting approaches achieve this co-localization by centrifugation or spinoculation of cells and virus. Techniques described herein improve transfection rates for both viral and non-viral vectors, while the device according to the invention is designed to create a homogeneous distribution of cells and vectors across the active area of the localization surface, without centrifugation, for example.

In general, according to one aspect, the invention features a viral transduction and/or electroporation device. The device comprises a membrane separating two chambers and two electroporation electrodes for the chambers. An electrical voltage source is provided for establishing an electrical field across the membrane and between the two electrodes. Electrophoresis electrodes can be added in some embodiments.

In general, according to another aspect, the invention features a viral transduction and/or electroporation system. The system comprises a membrane, electroporation electrodes, and an electrical voltage source for establishing an electrical field at the membrane and between the electrodes. In operation, one side of the membrane is loaded with cells, viruses for transduction, and cargo for electroporation and a fluid is flowed through the membrane to colocalize the cells, viruses and cargo against the membrane.

In one implementation, a first housing plate and a second housing plate define a first chamber on a first side of the membrane and a second chamber on a second side of the membrane. The first housing plate and the second housing plate function as the electroporation electrodes by either being conductive or having a conductive coating or conductive portion.

Preferably, an electroporation buffer reservoir and/or a cell media reservoir and/or a cell reservoir are provided. A pinning pump is used to flow fluid through the membrane. An unpinning pump is also helpful for flowing fluid through the membrane in the opposite direction to unpin the cells from the membrane.

Two electrophoresis electrodes can also be added.

The membrane may have a pore size of the membrane is less than 10 nanometers. A mesh can be also used to support the membrane against a flow of fluid.

In general, according to another aspect, the invention features a viral transduction and/or electroporation method. The method comprises loading one side of a membrane with cells, viruses for transduction, and cargo for electroporation, flowing fluid through the membrane to colocalize the cells, the viruses and the cargo against the membrane, and establishing an electrical field across the membrane to electroporate cells pinned to the membrane.

In general, according to another aspect, the invention features a viral transduction and/or electroporation system, comprising a membrane, electroporation electrodes, and an electrical voltage source for establishing an electrical field at the membrane and between the electrodes. Further provided is a means for tuning a distance between the electrodes and the membrane. In one example, a shim system is used.

In general, according to another aspect, the invention features a viral transduction and/or electroporation system, comprising a membrane separating two cavities, electroporation electrodes, an electrical voltage source for establishing an electrical field at the membrane and between the electrodes, an electroporation buffer reservoir for supplying electroporation butler into the cavities, a viral transduction reservoir for supplying viruses into the cavities, and a cell media reservoir for supplying cell media into the cavities.

In general, according to still another aspect, the invention features a viral transduction and/or electroporation system, comprising a membrane separating two cavities, electroporation electrodes, an electrical voltage source for establishing an electrical field at the membrane and between the electrodes, a pinning pump for pinning particles against the membrane, and an unpinning pump for unpinning particles from the membrane.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIGS. 1A and 1B are schematic cross-sectional views showing how the inventive dual-purpose viral transduction and electroporation device can function for both viral transduction (see FIG. 1A) and for electroporation (see FIG. 1B) by co-localizing target cells and vector on a porous polymer membrane. Both modes can be used sequentially, or even simultaneously. An alternative trapping modality is to use electrophoresis to co-localize cells and payload (cargo).

FIGS. 1C, 1D, and 1E are schematic diagrams illustrating the use of electrophoresis to drive charged molecules toward the porous membrane.

FIG. 2 is a scale exploded view of the parts making-up a dual-purpose viral transduction and electroporation device according to a first embodiment. The fluidic substrates are comprised of stainless steel, allowing them to also function as electroporation electrodes. O-ring gaskets and bolts clamp the structure together, sealing the fluidic chambers around a porous polymer membrane.

FIG. 3A is a cross-sectional side scale view and FIG. 3B is an exploded cross-sectional scale view of the dual-purpose viral transduction and electroporation device of the first embodiment. These views show how fluidic connections are made, and indicate nominal dimensions for critical features.

FIG. 4 is a cross-sectional side scale view of a dual-purpose viral transduction and electroporation device according to another embodiment.

FIGS. 5A and 5B are schematic cross-sectional views showing additional embodiments of the viral transduction and electroporation device.

FIG. 5C is a schematic plan view of another embodiment of the viral transduction and electroporation device.

FIG. 5D is a schematic cross-sectional view showing an additional embodiment of the viral transduction and electroporation device.

FIG. 5E is a schematic diagram showing yet another embodiment in which the viral transduction and electroporation device is provided with electrophoresis electrodes.

FIG. 6 is a block diagram of an automatic dual-purpose viral transduction and electroporation system employing the viral transduction and electroporation device.

FIGS. 7A, 7B, and 7C are flow diagrams showing different operations of the dual-purpose viral transduction and electroporation system.

FIGS. 8A and 8B are plots of transfection and transduction efficiency for different pinning flow fluxes.

FIG. 9 is a plot of transduction efficiency for different pinning flow fluxes.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The invention generally relates to approaches for transferring one or more material(s), referred to herein as “cargo” or “payload”, into cells. Examples of cargo materials include but are not limited to small molecules, chromosomes, DNA, RNA, (e.g., mRNA, siRNA, gRNA, ssRNA), other genetic materials, oligomers, biomarkers, proteins, transposons, biomolecule complexes, small molecules, therapeutic agents, and so forth.

As used herein, the term “transduction” refers to the introduction of recombinant viral vector particles into target cells. The term “transfection” refers to the forced introduction of small molecules (DNA, RNA, etc.) into cells, e.g., eukaryotic cells. Typically a non-viral method, transfection often involves electroporation, a technique that permeabilizes Opening transient pores or “holes” in the cell membrane allows the uptake of the cargo material and thus can alter or genetically modify the cells.

Aspects of the invention can be applied or adapted to transfer cargo to structures other than cells, such as, for example vesicles, liposomes, exosomes, micelles, and organelles.

In specific embodiments, the invention relates to a device in which it is possible to conduct both transduction and electroporation.

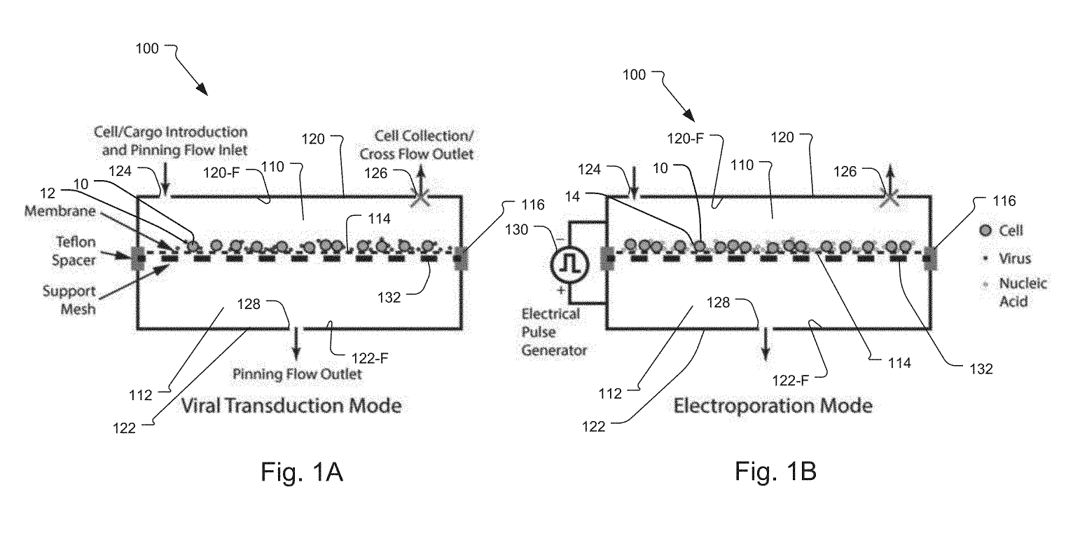

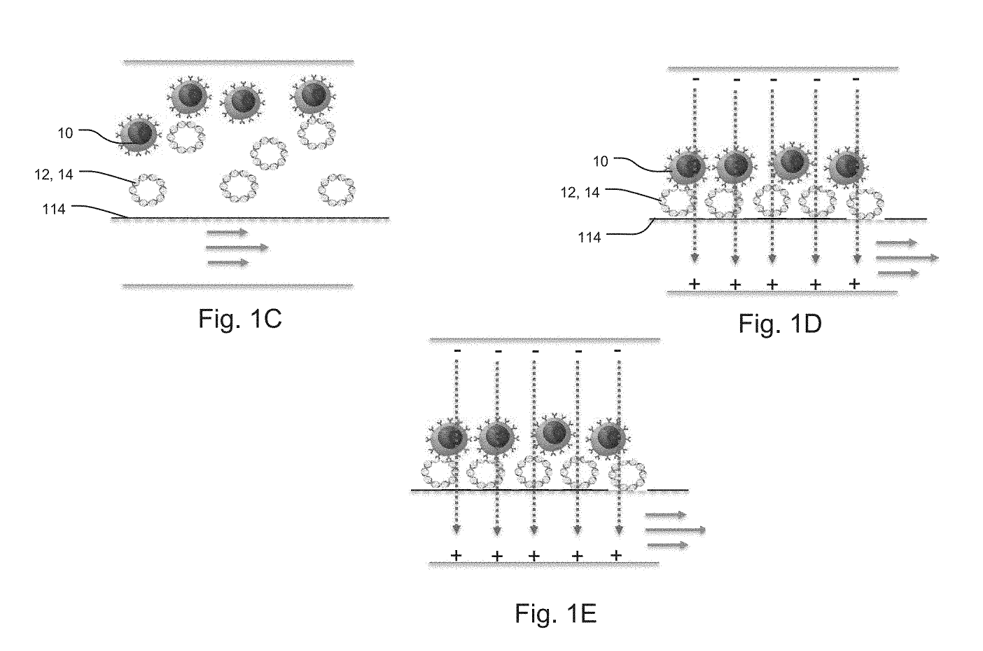

FIGS. 1A and 1B show a dual-purpose viral transduction and electroporation device 100, which is constructed according to the principles of the present invention.

The device 100 comprises two fluidic chambers 110, 112 separated by a semi-permeable membrane 114. The chambers are electrically isolated from each other. In the illustrated example, this isolation is achieved with an insulating dielectric spacer 116.

The fluidic chambers 110, 112 are defined by cavities in the two housing plates 120, 122, which are comprised of an electrically conductive material, such as stainless steel.

In this case, the housing plates 120, 122 function as two electroporation electrodes,

Alternatively, the housing may be comprised of a hard polymer, such as an acrylic (e.g., poly methyl methacrylate), and metal (e.g., platinum) thin film electrodes that may be deposited on the floor surfaces 120-F, 122-F of each of the top or first housing plate 120 and the bottom or second housing plate 122, respectively. Preferably, the material comprising the housing or thin film electrodes is non-reactive electrochemically, even passing Faradaic current.

In still other examples, the housing plates 120, 122 are fabricated from an electroactive polymer, such as PEDOT, which may be used to pass Faradaic current while minimizing electrolysis by-products.

The upper or first plate 120 will typically have a fluidic inlet port 124 and an outlet port 126 connecting to the upper or first fluid chamber 110, and clearance holes for screws.

The lower or second plate 122 has one or more fluidic ports such as inlet/outlet port 128 connecting to its fluid chamber, and tapped holes for screws.

The membrane 114 is often comprised of a polymer, such as polycarbonate or poly ether sulfone (PES), has thickness that ranges from 5 micrometers (μm) to 50 μm, and a pore size that is smaller than the size of the viral vectors or electroporation cargo used (typically <10 nanometers (nm)).

The dielectric spacer 116 has an annular shape and ranges in thickness from about 0.5 millimeters (mm) to 10 mm. It is often comprised of an insulative polymer material, such as PTFE.

A porous mesh 132 with very large perforations (allowing cells to pass through) is preferably added below the porous polymer membrane 114 for structural support. A nylon woven mesh, a glass fiber or cellulose filter, a metal mesh, or a porous conducting polymer layer such as polyacrylamide may be used as the mesh 132.

For viral transduction mode, shown in FIG. 1A, cells 10 and virus 12 are introduced into the upper fluidic chamber 110 through inlet 124. Flow of cell media is then initiated from an inlet in the top chamber, such that it passes through the membrane 114 and out inlet/outlet port 128 at the bottom chamber 112. Typical flow rates are in the range of 0.25-2 milliliters per minute (ml/min).

Because cells and virus or other electroporation cargo are too large to pass through the porous polymer membrane 116, they become pinned on the membrane 116 by the flow, and are effectively concentrated, increasing the number of interactions between them. This flow is maintained for the desired period of time (typically 30 minutes (min) to 24 hours), in one mode of operation.

Alternatively, cells and vector may be trapped against a membrane 116 using electrophoresis by applying an appropriate voltage across the membrane layer. This voltage may be applied using the electrodes for electroporation, or more preferably, separate electrodes located outside of the chamber 110 but connected electrically to the chamber via the liquid flow paths to minimize bubble formation in the chamber. This is illustrated in FIGS. 1C, 1D and 1E. In more detail, FIG. 1C shows the flow of structures, e.g., cells 10, etc., and viruses or cargo 12, 14 in the absence of electric forces. As seen in FIG. 1D, electrically charged virus or cargo molecules 12, 14 and/or cells/structures 10 can be directed toward the membrane 114 (for enhanced contact) by electrophoresis (the movement of charged particles in a fluid or gel under the influence of an electric field). Permeabilization of the cells/structures 10 is then conducted by electroporation (FIG. 1E).

Cells are removed from the device 100 by simultaneously actuating flow through the inlet port 124 and reversing flow through the inlet/outlet port 128 such that fluid now flows into the top fluidic chamber 110 through inlet port 124 and flows into the bottom fluidic chamber 112 through the inlet/outlet port 128. As a consequence, flow exits the outlet port 126 in the top chamber 110.

In some embodiments, cells exiting the outlet port are then introduced into a second apparatus that contains a membrane with a pore size larger than that of the vector, but smaller than the cell diameter (0.5 μm-2 μm) to separate vector from cells.

For electroporation mode, shown in FIG. 1B, cells 10 are introduced with cargo 14 (DNA, gRNA, mRNA, siRNA, ssRNA, RNP, transcription factors, protein components for CRISPR modification etc.) and then are pinned onto or against the membrane 116 as described above for the viral case. Cargo can also be introduced after the cells have been pinned.

An alternating current (AC) (for example, sinusoids or pulse trains with periods/pulse widths ranging from 10 nanoseconds (ns) to 100's of microseconds) or direct current (DC) voltage is applied to the electrodes and across the plates 120, 122 by the electrical function generator voltage source 130. For square-wave pulse trains, typically 1-5 pulses are applied. The pulse trains may be either monophasic or biphasic. The voltage is tuned such that the magnitude of the resulting electric field in the solution is sufficient to achieve permeabilization in the given cell type, and is typically in the range of 2-200 kV/m.

Cells are then removed from the device as described above.

In some embodiments, cells are introduced into a second apparatus containing a membrane with a larger pore size, as described above, to remove non-integrated cargo, as described above.

Finally, electroporation and viral transduction can be conducted sequentially using this device 100.

In more detail, after cells have been electroporated as described above in connection with FIG. 1B, virus can be introduced into one of the inlets 124, in the top half of the device 100, and transduction can be carried out as described above in connection with FIG. 1A. Virus particles can also be introduced along with the transfection cargo, so that they are present during the electroporation event, enabling electroporation-assisted viral transduction.

Conversely, electroporation can follow viral transduction, provided that sufficient time has passed between the two events (at least 24 hours).

For both electroporation and viral transduction, fluid is perfused from the top chamber 110 through the membrane 116 and out of the bottom inlet/outlet port 128. This imparts a constant downward drag force on particles (cells, virus, and nucleic acids) that are necessary to co-localize and concentrate the particles on the membrane 116 to increase interactions.

The device also enables switching of fluid properties, including conductivity, density,and protein composition to allow for rapid buffer exchanges that can enhance or affect the different viral transfer processes or transfection processes. For example, a low conductivity buffer is beneficial for electroporation in order to limit electrolysis product and. Joule heating, but can negatively affect cell health. In this case, a low conductivity buffer may be perfused briefly during the electroporation event, and then a more optimal buffer can be perfused to support cell health and viability.

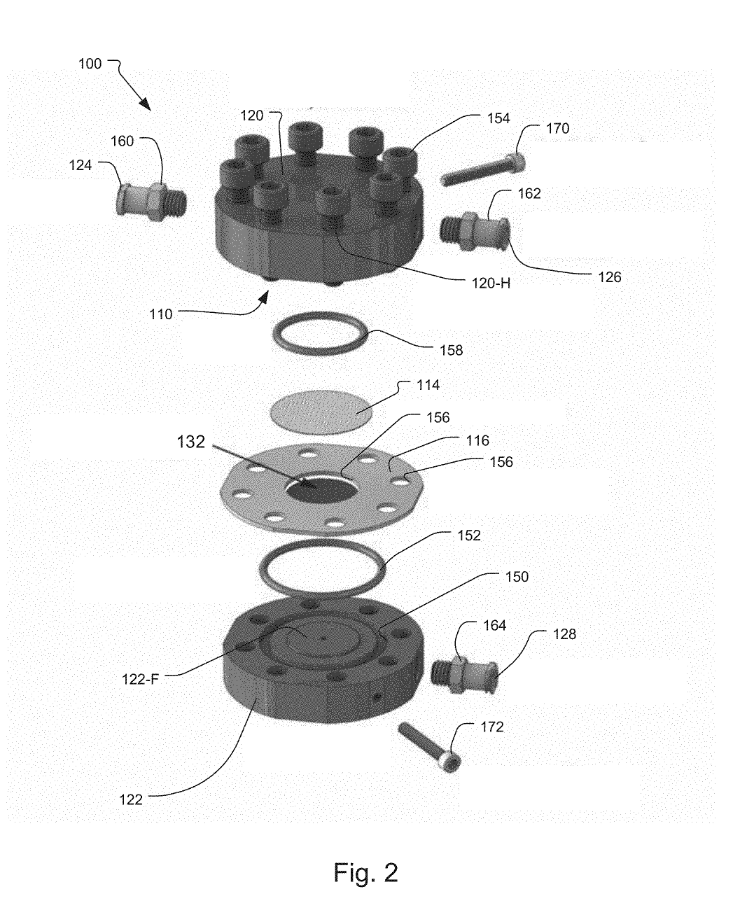

FIG. 2 is a perspective scale exploded view of one embodiment of the dual-purpose viral transduction and electroporation device 100.

The two plates 120 and 122 are bolted together using nylon screws. Each of the plates is generally disk shaped with eight holes 120-H, 122-H extending in the axial direction of the plates around the circumference of the plates. The holes 120-H, 122-H receive the non-conductive nylon screws 154.

The inner side of the lower plate 122 has an annular O-ring trench 150 for receiving a lower O-ring 152. At the center of the bottom plate is the floor surface 122-F.

The electrically insulating spacer 116 separates the two plates 120, 122. Around its circumference there are eight holes matching the holes 120-H, 122-H of the two plates 120, 122. The spacer 116 has a center hole 156. In the illustrated embodiment the center hole 156 is circular and has a diameter of less than several (2 or 4) centimeters. Its radius is 8 mm in the illustrated example. In one implementation, the spacer is fabricated from polytetrafluoroethene (PTFE) (Teflon) or possibly perfluoroalkoxy polymer resin (PFA).

The membrane 114 is supported by the spacer 116 and extends over the center hole 156 of the spacer 116. Possibly a nylon mesh 132 is provided under the membrane 114 and also across the center hole 156 at the center of the Teflon spacer 116 in order to support the membrane 114. The lower O-ring 152 and an upper O-ring 158 ensure a fluid tight connection between the plates 120 and 122 and either side of the Teflon spacer 116.

In some embodiments, the lower O-ring 152 and/or the upper O-ring 158 are replaced with two gaskets on either side of the spacer 116,

In the present embodiment, the membrane 114 has a molecular cutoff of less than 200 kiloDaltons. (kDa) and thus only passes particles having a molecular mass of less than 200 kDa. In the preferred embodiments, the cutoff is 1.00 kDa or less or 50 kDa or less and has a thickness of about 0.15 mm. The mesh 132 has 40-50% open area and has a thickness of about 0.3 mm.

Coupling to the inlet port 124 and the outlet port 126 is provided by corresponding Luer Lok threaded connectors 160, 162 that are screwed into the body of the upper plate 120. In a similar vein, coupling to inlet/outlet port 128 is provided by another Luer Lok threaded connector 164 that is screwed into the bottom plate 122.

Electrical connections to each of the upper plate 120 and the lower plate 122 are provided, respectively, by metal (stainless steel) bolts or screws 170, 172. These screws are bolted into threaded holes in the upper plate 120 and the lower plate 122.

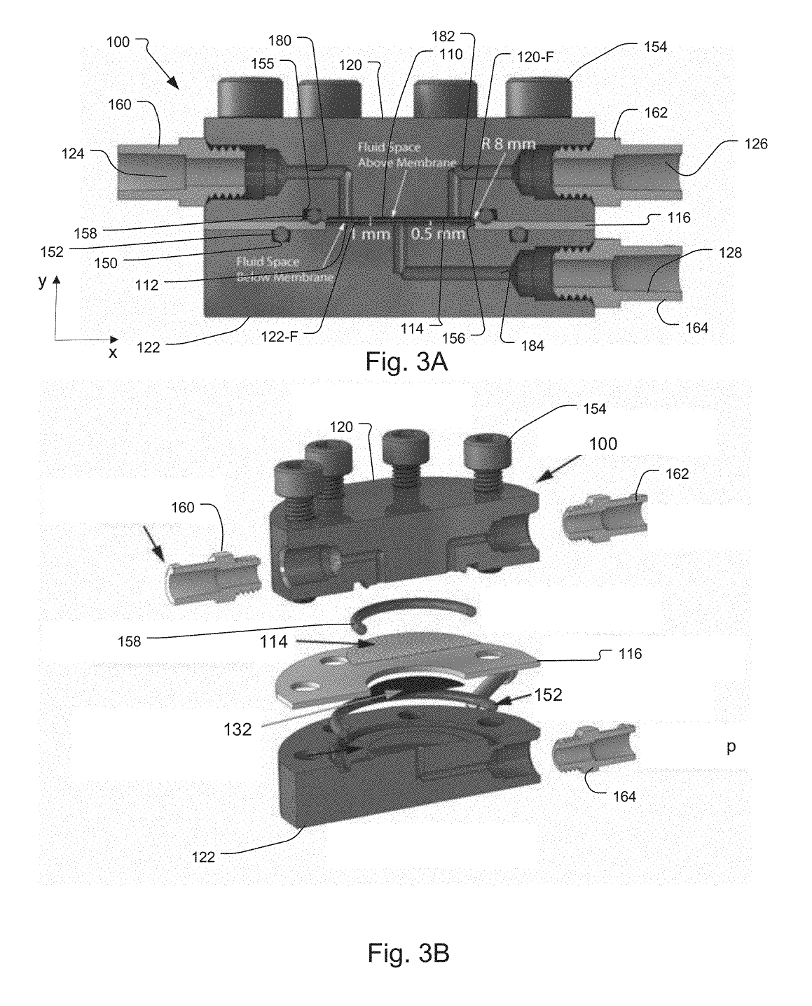

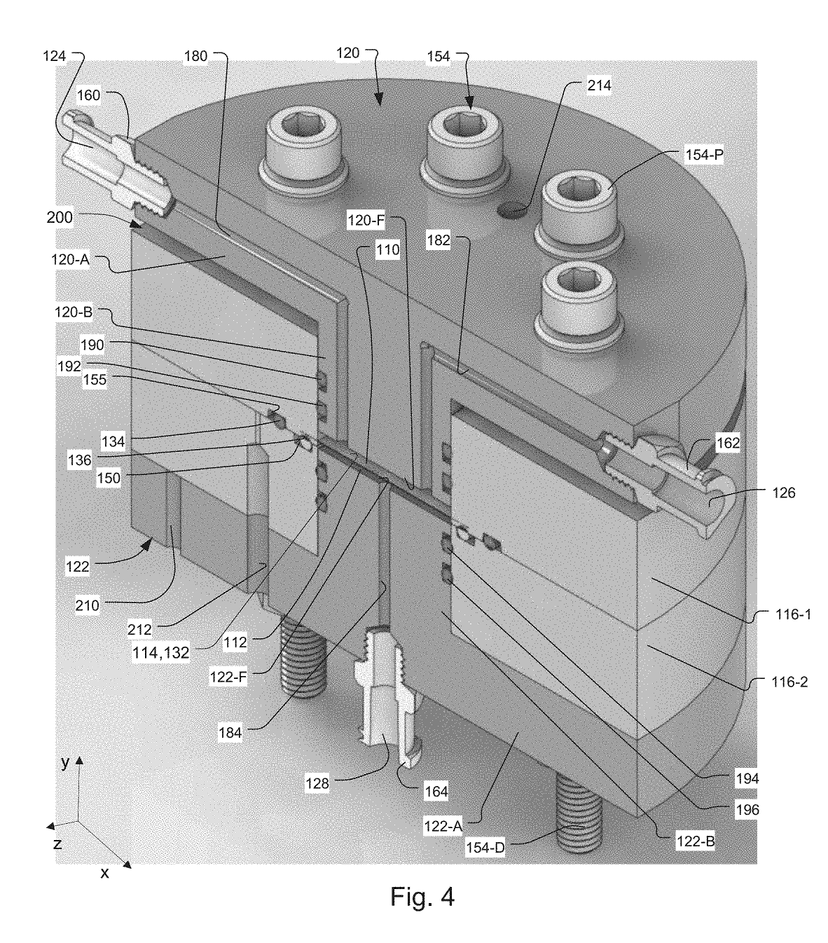

FIG. 3A shows a cross-sectional view of the device 100.

It further shows a series of fluid flow channels formed through the body of the upper plate 120 and the lower plate 122. These provide a manifold for fluid communication between the ports 124, 126, and 128 and the upper chamber 110 and the lower chamber 112. In the present embodiment, each of the plates 120, 122 is fabricated from stainless steel. Other materials can be employed.

In more detail, a top input channel 180 formed through the body of the top plate 120 provides a fluid flow path between the input port 124 and the top chamber 110. An upper output channel 182 formed through the body of the top plate 120 provides a fluid flow path between the top chamber 110 and the output port 126. A lower outlet channel 184 formed through the body of the bottom plate 122 provides a fluid flow path between the input/output port 128 and the bottom chamber 112.

In terms of the dimensions of the top chamber 110 and the bottom chamber, the height of each chamber, the y-axis distances between the respective floor surfaces 120-F, 122-F and the membrane 114, are preferably less than several (2 or 4) millimeters. In the illustrated example, the distances are each about 0.5 mm. The distance between the floor surfaces 120-F, 122-F and also the thickness of the spacer 116 are less than 4 or 8 millimeters, about 1 mm in the illustrated embodiment.

The view in FIG. 3A better show the O-ring trenches for each of the upper O-ring 158 and the lower O-ring 152. Specifically, the lower O-ring 152 sits in the annular O-ring trench 150 formed in the lower plate 122 and seals against the lower face of the spacer 116. The upper O-ring 158 sits in an annular O-ring trench 155 formed in the upper plate 120 and seals against the upper face of the spacer 116.

Finally, more detail is shown in the cross-sectional exploded view of FIG. 3B.

FIG. 4 shows another embodiment that enables the adjustment of the size of the upper or first fluidic chamber 110 and the lower or second fluidic chamber 112.

In more detail, the upper or first housing plate 120 is generally similar functionally to the previous embodiment. However, it comprises a flange portion 120-A and a piston portion 120-B. The flange portion 120-A is generally cylindrical with a squat aspect ratio. The piston portion 120-B is preferably unitary with the flange portion 120-A and is also cylindrical and coaxial with the flange portion 120-A.

The lower or second housing plate 122 similarly comprises a flange portion 122-A with a coaxial piston portion 122-B.

The upper input channel 180 is bored through the flange portion 120-A and then extends through the piston portion 120-B to terminate at the floor surface 120-F of the piston portion 120-B of the top plate 120. It provides a fluid flow path between the input port 124 and the upper chamber 110.

The top output channel 182 begins at the floor surface 120-F of the piston portion 120-B of the top plate 120, extends through the piston portion to the flange portion 120-A and then terminates at the output port 126. It provides the fluid flow path out of the top chamber 110.

The bottom inlet/outlet channel 184 begins at the floor surface 122-F of the piston portion 122-B of the lower plate 122, extends through the piston portion to the flange portion 122-A and then terminates at the input/output port 128. It provides a fluid flow path between the bottom chamber 112 and port 128.

The spacer and electrically isolation between the two plates 120, 122 takes the form of two annular spacers, i.e., an upper insulating dielectric half spacer 116-1 and a lower insulating dielectric half spacer 116-2. The piston portion 120-B of the upper plate 120 extends through the center hole of the upper half spacer 116-1; and the piston portion 122-B of the lower plate 122 extends through the center hole of the lower half spacer 116-2.

Two upper piston O-rings 190, 192 provide a seal between the outer surface of the piston portion 120-B of the upper plate 120 and the inner wall of the center hole of the upper insulating dielectric half spacer 116-1,

Two lower piston O-rings 194, 196 provide a seal between the outer surface of the piston portion 122-B of the lower plate 122 and the inner wall of the center hole of the lower insulating dielectric half spacer 116-2.

A shim slot 200 exists between the lower surface of the flange portion 120-A of the upper plate 120 and the upper surface of the upper half spacer 116-1. This space can be filled with possibly a wedge-shaped shim or an annular-shim shim. The thickness of this shim used in the shim slot 200 is used to control the degree to which the piston portion 120-B projects through or is recessed within the upper half spacer 116-1 and thus the size of the upper or first fluidic chamber 110. The size of the lower or second fluidic chamber 112 is then controlled by the relative vertical thicknesses (y-axis direction) of the upper spacer 116-1 and the lower spacer 116-2 since the membrane 114 and optional mesh 132 are sandwiched between the lower surface of the upper half spacer 116-1 and the upper surface of the lower half spacer 116-2. The membrane is secured between the two dielectric spacers with O-rings for fluidic seals and bolts. Shim thicknesses from 0.5 mm to 3 mm were used on the upper and lower chambers to create corresponding chamber heights of 0.5 to 3 mm.

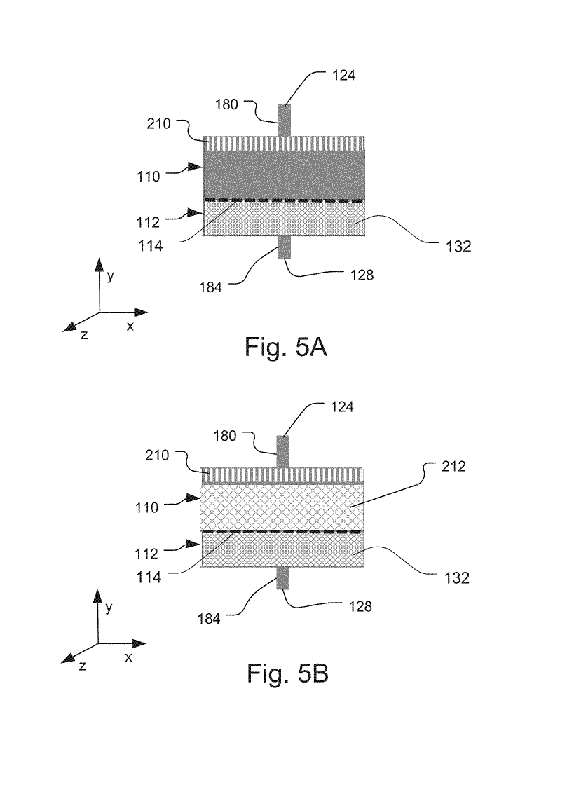

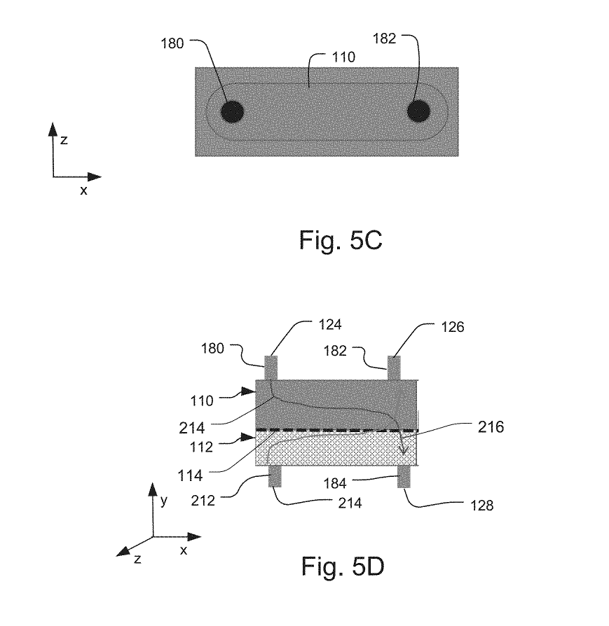

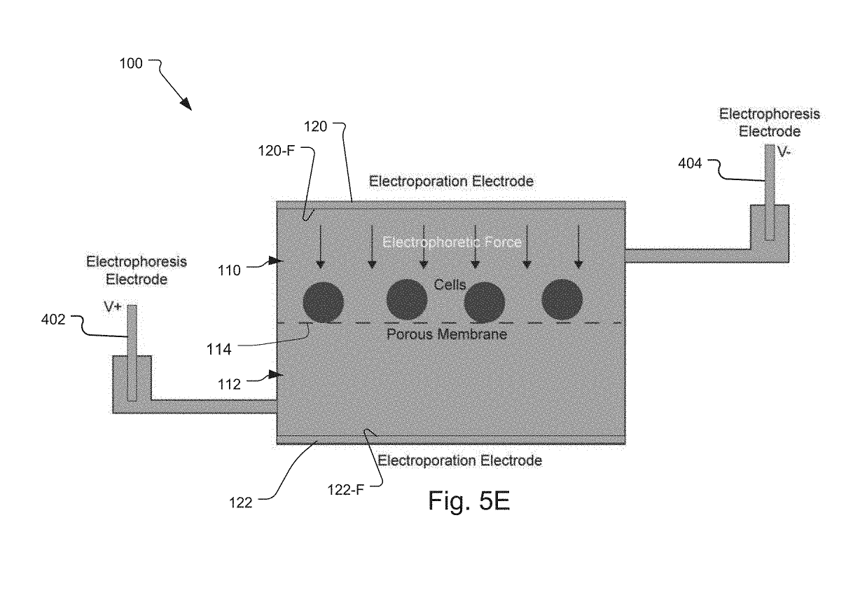

FIG. 5A shows another possible configuration of the upper and lower fluidic chambers 110, 112.

Here, the top input channel 180 through the body of the top plate ends in a diffuser or manifold 210 that distributes the fluid flow into the top chamber laterally in both the x and z directions to create an evenly distributed flows.

Moreover, in this example, the nylon mesh 132 is provided under the membrane 114 and fills the lower chamber 112 to thereby evenly support the membrane, thus flattening it in the x-z plane even as media flows through it.

FIG. 5B shows another possible configuration of the upper and lower fluidic chambers 110, 112.

Here, porous top support 212 is provided above the membrane 114 and fills the upper chamber 110 to thereby evenly support the membrane to render it flatter and prevent bowing in the x-z plane even as media flows through it in the reverse direction either out the channel 180 or a separate upper output channel 182.

FIG. 5C shows another possible configuration of the upper and lower fluidic chambers 110, 112.

In the previous designs, the chambers were circular in the x/z plane. In the example, the chambers are oval.

In addition the chambers can be either fluidically sealed using O-rings as in the previous configurations, or with a gasket which minimizes void spaces where cells and other materials can get trapped.

FIG. 5D shows another possible configuration of the upper and lower fluidic chambers 110, 112.

As in the previous designs, fluid flows into the upper chamber via the inlet port 124 and the upper input channel 180. Fluid is then withdrawn from the lower chamber 112 via the lower outlet channel 184.

This design, however, further has a lower input channel 212 or which fluid can be injected into the lower chamber 112.

As in previous examples, fluid is removed from the upper chamber 110 via the upper output channel 182.

Thus, when the cells are to be pinned against the membrane 114, fluid flows according to arrow 214. In contrast, when the cells are to be unpinned from the membrane 114, fluid flows according to arrow 216.

Further embodiments relate to the use of electrophoresis to drive electrically charged cells or other structures towards the membrane 114. Shown in FIG. 5E, for instance, is device 400 including membrane 114 and electroporation electrodes, plates 120, 122, for example. Device 100 also includes electrophoresis electrodes 402, 404 for applying an electrical field suitable for moving charged structures toward the membrane 114. In FIG. 5E, these electrodes are remote. Other arrangements can be employed, however. Larger than the membrane pores, the structures are pinned, trapped, retained, etc. at the surface of the membrane.

Cargo 14 is brought into close proximity to or into contact with the structures 10 (e.g., cells) by a suitable technique such as, for example electrostatic forces, electrophoresis, a membrane pore structure that prevents the passage of payload materials, and so forth. In some implementations, the cargo is not immobilized at the membrane surface but rather manipulated in dynamic fashion. If an electrophoretic voltage is employed to move the cargo, it can be applied before, simultaneously or after application of the voltage used to pin the structures (cells) at the membrane 114.

In cases in which transport of cells and/or cargo toward membrane 114 is enhanced electrophoretically, the electrical field applied can be within the range of from about 10 to about 70 volts per centimeter (V/cm), e.g., 30-40 V/cm, 40-55 V/cm or 55-70 V/cm. Electrodes 402, 404 can be configured to generate a mobility of about 3 μm/sec/V/cm, such as 0.5-2, 2-5, or 5-10 μm/sec/V/cm.

As described with reference to FIGS. 1C through 1E, electroporation can then be used to permeabilize the cells and make possible the uptake of the cargo. The electroporation voltage can be applied in pulses. Its magnitude often exceeds that of voltages typically used to implement the electrophoresis operation.

Bringing structures and cargo in close proximity, using electrophoresis, for example, facilitates the uptake of the cargo once the structures are permeabilized.

Additional modifications are possible. For example, the polymer membrane can be bound or affixed to a stiff intermediate layer or directly to the underlying porous support for increased rigidity. Also, multiple inlet and outlet ports can be designed on the lower chamber as well for increasing fluid routing for distributing cells for pinning and unpinning.

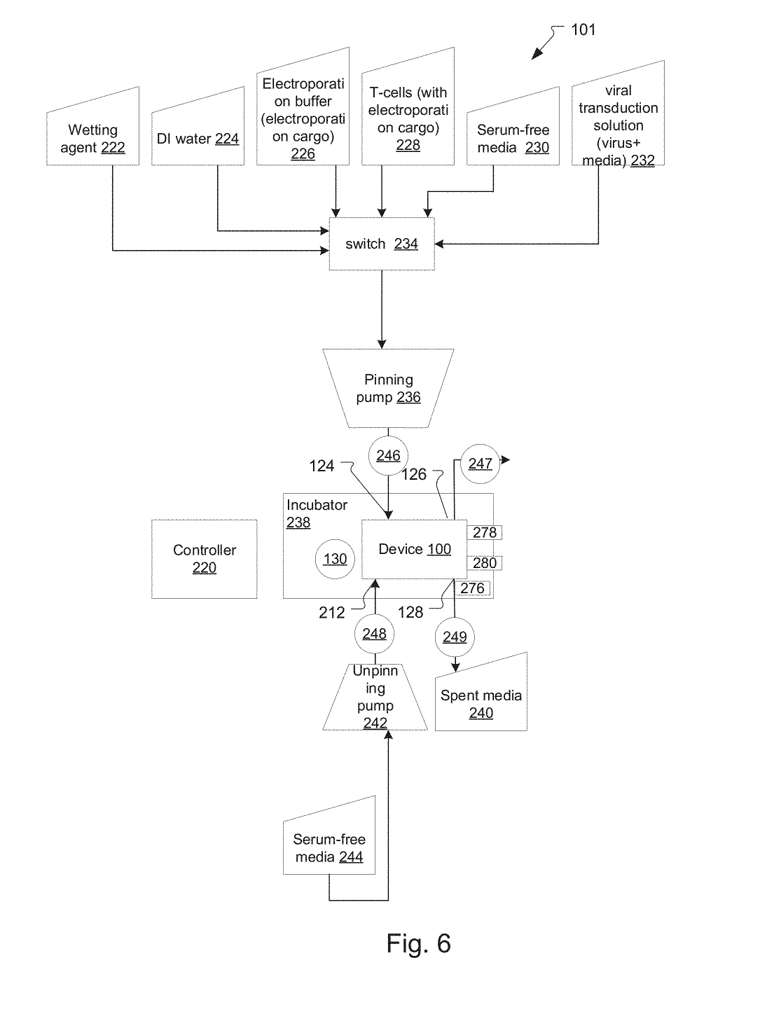

FIG. 6 is a schematic diagram showing the device 100 integrated into an automatic dual-purpose viral transduction and electroporation system 101.

In more detail, the system 101 comprises a series of reservoirs. In the illustrate embodiment, there are the following reservoirs: 1) a wetting agent reservoir 222, 2) a deionized (DI) water reservoir 224, 3) an electroporation buffer reservoir (possibly also containing an electroporation cargo) 226, 4) a T-cell reservoir 228 (possibly also containing an electroporation cargo), 5) a serum-free cell media reservoir 230, and 6) a viral transduction solution reservoir 232 (containing viruses and media).

Each of these reservoirs supply their respective fluid to a switch 234 that is controlled by a controller 220. The switch selectively provides the fluid from one of the reservoirs to a pinning pump 236 that flows the selected fluid into the device 100 via the inlet port 124 at a fluid flow rate dictated by the controller 220.

The fluid flowing out of the device 100 is provide to the spent media reservoir 240 via the lower chamber outlet port 128. An unpinning fluid flow is provided by supplying fluid from the serum-free cell media reservoir 244 to an unpinning pump 242 to flow the fluid into the device under the control of the controller 220.

Serum-free cell media contained in the reservoir 230 is a cell culture media that promotes the health and growth of the cells. On the other hand, the electroporation buffer contained in the reservoir 226 generally differs from the serum-free cell media in terms of how long the cells can survive in the respective buffers. Serum free media typically contains physiological salt concentrations that match cell osmolarity and nutrients. On the other hand, an example of an electroporation buffer would be BTX low-conductivity buffer (sold by BTX). Such electroporation buffer typically has lower salt concentration to reduce the conductivity, but has added sugars to reduce osmotic shock to the cells.

Further provided are valves 246, 247, 248, and 249, which are controlled by the controller 220 to further control how fluid leaves the device.

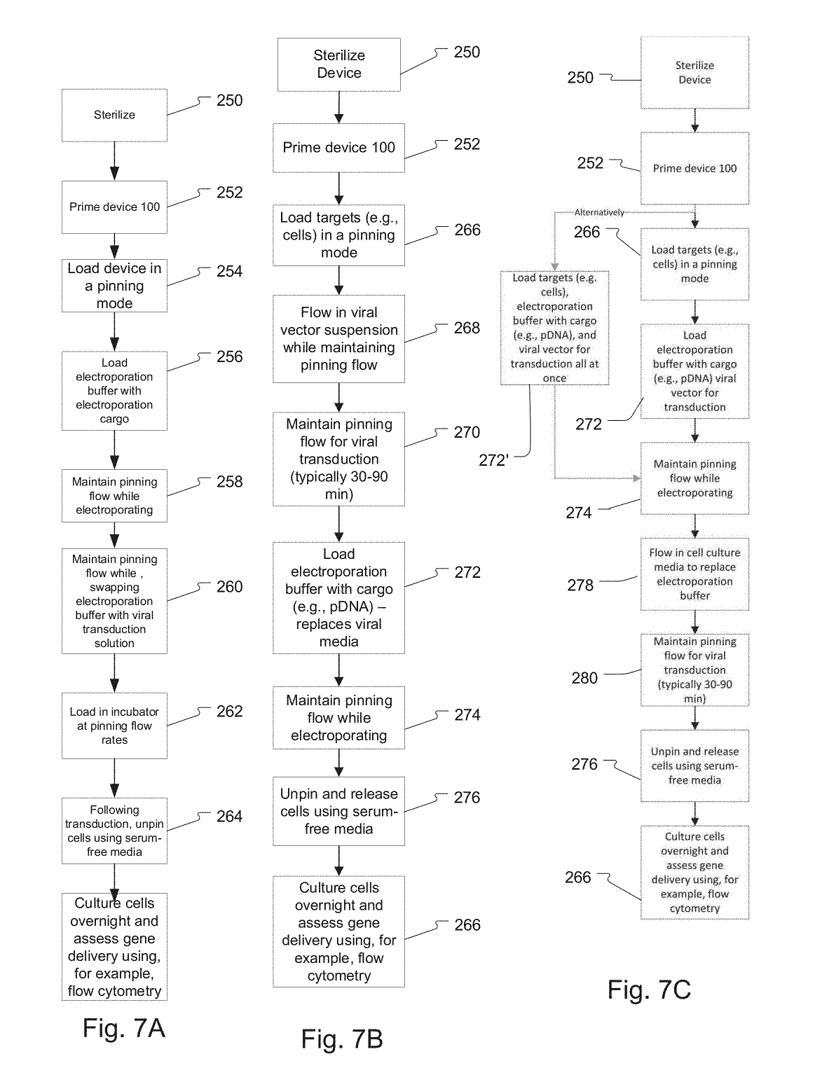

FIG. 7A is a flow diagram showing the operation of the device 100 by the system 101.

Typically, the device 100 is first assembled and then sterilized, e.g., by using ethylene oxide, in step 250. Often, a 12 hour sterilization cycle and followed by a 72 hour de-gas in vacuum is adequate.

Then the device is connected into the system 101.

The controller 220 first primes the device 100 with a wetting agent (such as ethanol) to remove air bubbles in step 252. With reference to FIG. 6, the switch 234 connects the wetting agent reservoir 222 to the pinning pump 236. Valves 246 and 249 are opened, and the controller 220 activates the pinning pump 236.

Then, the controller 220 removes the ethanol by controlling the switch 234 to connect the DI water reservoir 224 to the pinning pump 236. Valves 246 and 249 are opened, and the controller activates the pinning pump 236.

Next the controller 220 fills the device with electroporation buffer (e.g., Gemini BTX) by controlling the switch 234 to connect the electroporation buffer reservoir 226 to the pinning pump 236. Valves 246 and 249 are opened, and the controller activates the pinning pump 236.

In a different example, the controller 220 fills the device with serum-free media by controlling the switch 234 to connect the serum-free media reservoir 230 to the pinning pump 236. Valves 246 and 249 are opened, and the controller activates the pinning pump 236.

The controller then loads the device 100 in a pinning mode where the flow rate is, for example 100 microliters (μL)/min to 1 milliliter (mL)/min in step 254. In one example, activated T-cells either in electroporation buffer (with or without electroporation cargo such as mRNA) or a serum-free media are selected by the switch 234 connecting to the T-cells reservoir 228. Valves 246 and 249 are opened, and the controller activates the pinning pump 236.

In pinning mode, electroporation buffer (with electroporation cargo) is loaded into the device in step 256. The cargo is loaded onto the membrane since it is too large to flow through it. The controller 220 continues filling device 100 with electroporation buffer until the media is exchanged across the membrane 114 such that the effluent conductivity at the pinning outlet port 128 closely matches the value of bulk buffer (BTX is typically 0.08 mS/cm), implying or indicating that media has been completely exchanged. This is detected by the controller 220 monitoring a conductivity sensor 276 that detects the conductivity of the fluid at exit port 128. The controller 220 further modulates the operation of the pinning pump and its flow rate by monitoring the pressure in the device via an upper chamber pressure transducer 278 (measuring the pressure in the upper chamber 110) and lower chamber pressure transducer 280 (measuring the pressure in the lower chamber 112).

The controller then initiates electroporation by activating the function generator/voltage source 130 in step 258. In one example, the controller 220 drives the electroporation device 100 by pulsing a 0.6 ms pulse of field strength 155-175 V/mm. During this operation, the pinning pump continues to operation to colocalize the cells with the cargo on the membrane.

While in pinning mode, the controller 220 swaps electroporation buffer with viral transduction solution (virus+media) by controlling the switch 234 to now connect the pinning pump 236 to the viral transduction solution reservoir 232 in step 260. This loads viruses onto the membrane since they are too large to flow through it. Pinning flow rates are maintained to colocalize the cells with the viruses on the membrane.

At this point, the controller 220 preferably activates an incubator 238 in which device 100 is contained in step 262. The controller 220 maintains pinning flow rates of between 20-100 μL/min (across a membrane that is 1″ in diameter). Pinning flow for transduction operation is maintained for a suitable length of time, 1.5 hours, for instance.

Following transduction, the controller 220 unpins the cells by closing valves 246 and 249, opening valves 248 and 247 and operating the unpinning pump 242 to flow serum-free media at high speeds (10-50 μL/min top, and 1 mL/min bottom) through the device 100 to release cells from membrane surface and collect these cell leaving port 126 in step 264.

At this point, the cells are cultured overnight and flow cytometry can be conducted 24 hours later to identify cells that are transfected and transduced in step 266.

In a different operation, the system 101 could be run in reverse with transduction first then transfection or also by loading viral vector+mRNA simultaneously, electroporating, then transducing for possible enhanced performance.

FIG. 7B is a flow diagram showing an operation of the device 100 by the system 101.

As before, the device is sterilized and primed in steps 250 and 252.

Next, the controller 220 loads the device 100 with the targets, e.g., T cells, in a pinning mode in step 266. Here, the controller 220 controls the switch to connect the T-cells reservoir 228 to the pinning pump 238.

Next, step 268, the controller 220 loads the device with a viral vector suspension while maintaining a pinning flow rate. Here, the controller 220 controls the switch to connect a reservoir containing the viral vector suspension to the pinning pump 238.

Next, step 270, the controller 220 maintains a pinning flow for viral transduction such as for 30 to 60 minutes. Here, the controller 220 controls the switch 234 to connect to the serum-free media reservoir 230 to maintain cell viability.

Next the controller 220 fills the device with electroporation buffer (e.g., Gemini BTX). The pDNA, for example, is in or is added to the buffer in step 272. Here, the controller 220 connects the electroporation buffer reservoir 226 to the pinning pump 236. Valves 246 and 249 are opened, and the controller activates the pinning pump 236.

In step 274, the controller maintains a pinning flow while then initiating electroporation by activating the function generator/voltage source 130.

In step 27 6, the cells are unpinned and released using the serum-free media from the reservoir and provided by the pump 242.

At this point, the cells are cultured overnight and flow cytometry can be conducted 24 hours later to identify cells that are transfected and transduced in step 266.

FIG. 7C is another flow diagram showing an operation of the device 100 by the system 101.

As before, the device is sterilized and primed in steps 250 and 252.

Next, the controller 220 loads the device 100 with the targets, e.g., T cells, in a pinning mode in step 266. Here, the controller 220 controls the switch to connect the T-cells reservoir 228 to the pinning pump 238.

Next, step 268, the controller 220 loads the devices with a viral vector suspension while maintaining a pinning flow rate. Here, the controller 220 controls the switch to connect a reservoir containing the viral vector suspension to the pinning pump 238.

Next the controller 220 fills the device with electroporation buffer (e.g., Gemini BTX). The pDNA, for example, is in or is added to the buffer in step 272. Here, the controller 220 connects the electroporation buffer reservoir 226 to the pinning pump 236. Valves 246 and 249 are opened, and the controller activates the pinning pump 236.

In another flow, the targets, electroporation buffer with cargo and viral vector for transductions are loaded all at once in step 272′.

In step 278, the controller flows cell culture media to replace the electroporation buffer. In step 280 the controller maintains a pinning flow for viral transduction for 30 to 90 minutes, for example.

In step 276, the cells are unpinned and released using the serum-free media from the reservoir and provided by the pump 242.

At this point, the cells are cultured overnight and flow cytometry can be conducted 24 hours later to identify cells that are transfected and transduced in step 266.

In general, the controller 220 can be used to calculate the field strength of the electroporation by measuring the device resistance and monitoring the fluid conductivity using the conductivity sensor 276.

When assembled, the device is preferably calibrated. The entire device 100 is filled with a solution of known conductivity (electroporation buffer or media) and a resistance measurement is conducted using electrochemical impedance spectroscopy (EIS). This gives a baseline resistance for the device. As the device is then loaded with cells, EIS measurements can continue to be obtained to calculate the final resistance in the top chamber where cells are loaded so that the field strength of the electroporation is ultimately known.

The effluent conductivity of the bottom port can be monitored using conductivity sensor 276 to determine that media has been fully exchanged in the device and minimize the presence of anything else in the media which would inhibit transduction/transfection

Finally, the controller can also collect data regarding the pressure drop across the membrane 114 by monitoring the pressure measured in each of the chambers 110, 112 by the inline pressure sensors on the top outlet port 126 and the inlet or outlet port 128, 212 using respective pressure transducers 278, 280 to characterize changes in pressure due to cell loading on the membrane 114 and pinning fluxes.

According to one experiment, 2 million activated T-cells were loaded at 2 million cells/mL with 50 ug/mL mCherry mRNA.

The cells were washed through pinning in BTX media until the effluent pinning conductivity reached a minimum (usually ˜0.1 mS/cm)

The device 100 was electroporated at a target field strength of 175 V/mm with a single pulse of width 0.6 msec. The applied voltage was calculated by measuring the resistance of the device loaded with cells and comparing to a curve fit prepared for calibration.

Following electroporation the cells were exchanged with texmacs and AAV virus (2 uL or a titer of 10\hat{ }10 vg/ul). They were then transduced for 90 minutes.

They were then released and cultured overnight.

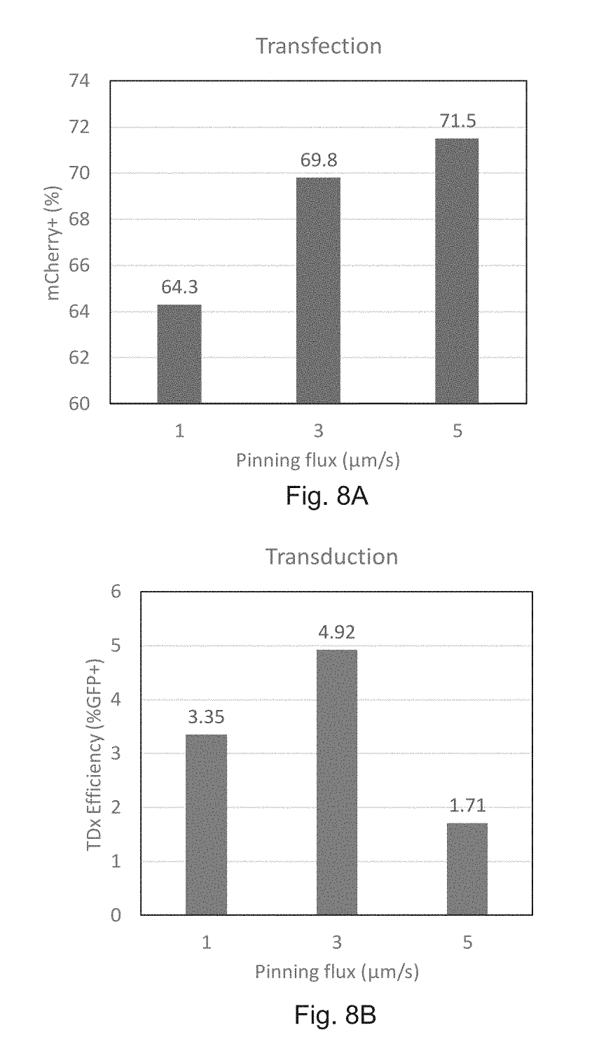

FIGS. 8A and 8B are plots of transfection and transduction efficiency for different pinning flow fluxes. Specifically, in the experiment, 2×10⁶ activated T-cells were electroporated at 1.75 kV/cm field strength in the presence of 50 ug/mL mCherry mRNA. Following electroporation AAV virus was loaded and cells were transduced at varying pinning fluxes between 1-5 μm/s (flow rates between 20-100 μL/min) across the membrane 114. This pinning enhances co-localization of the virus and cells enhancing transduction. Results show both successful transfection and transduction of the cells.

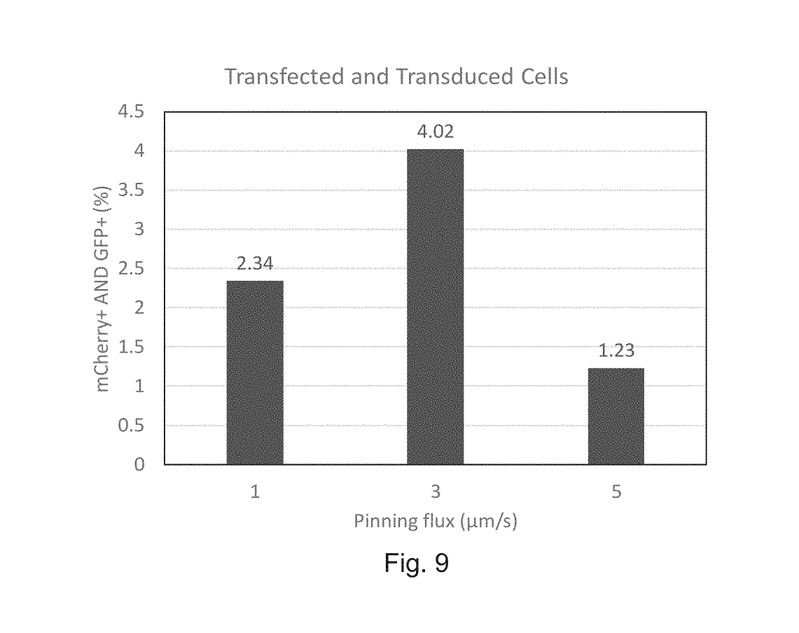

FIG. 9 is a plot of transduction efficiency for different pinning flow fluxes. In this experiment, 2×10⁶ activated T-cells were electroporated at 1.75 kV/cm field strength in the presence of 50 ug/mL mCherry mRNA. Following electroporation AAV virus was loaded and cells were transduced at varying pinning flow rates between 1-5 μm/s (20-100 uL/min across the membrane). This pinning increased co-localization of the virus and cells, enhancing transduction.

This device demonstrated a population of activated T-cells that are both transfected and transduced, showing up to 4% double positive cells.

While this invention has been particularly shown and described with references to preferred embodiments thereof, it will be understood by those skilled in the art that various changes in form and details may be made therein without departing from the scope of the invention encompassed by the appended claims.

What is claimed is:

1. A viral transduction and/or electroporation system comprising:
a membrane;
electroporation electrodes; and
an electrical voltage source for establishing an electrical field at the membrane and between the electrodes,
wherein one side of the membrane is loaded with cells, viruses for transduction, and cargo for electroporation and fluid flowing through the membrane colocalizes the cells, viruses and cargo against the membrane.

2. The system of claim 1, further comprising a first housing plate and a second housing plate defining a first chamber on a first side of the membrane and a second chamber on a second side of the membrane, wherein the first housing plate and the second housing plate function as the electroporation electrodes.

3. The system of claim 1, further comprising an electroporation buffer reservoir for providing electroporation buffer for flowing through the membrane.

4. The system of claim 1, further comprising a cell media reservoir for providing cell media for flowing through the membrane.

5. The system of claim 1, further comprising a cell reservoir for providing cells for loading on the membrane.

6. The system of claim 1, further comprising a pinning pump for flowing fluid through the membrane.

7. The system of claim 1, further comprising an unpinning pump for flowing fluid through the membrane in the opposite direction to unpin the cells from the membrane.

8. The system of claim 1, further comprising two electrophoresis electrodes.

9. The system of claim 1, wherein a pore size of the membrane is less than 10 nanometers.

10. The system of claim 1, further comprising a mesh for supporting the membrane against a flow of fluid.

11. A viral transduction and/or electroporation method, comprising:
loading one side of a membrane with cells, viruses for transduction, and cargo for electroporation;
flowing fluid through the membrane to colocalize the cells, the viruses and the cargo against the membrane; and
establishing an electrical field across the membrane to electroporate cell pinned to the membrane.

12. The method of claim 11, further comprising flowing an electroporation buffer through the membrane.

13. The method of claim 11, further comprising flowing a cell media through the membrane.

14. The method of claim 11, further comprising flowing fluid through the membrane in the opposite direction to unpin the cells from the membrane.

15. The method of claim 11, further comprising performing electrophoresis at the membrane.

16. The method of claim 11, further comprising electroporating cells on the membrane while flowing electroporation buffer through the membrane.

17. The method of claim 16, further comprising, after electroporating the cells, loading the membrane with viruses.

18. The method of claim 17, further comprising after electroporating the cells but before loading the membrane with viruses, flowing cell media through the membrane.

19. The method of claim 16, further comprising prior to electroporating cells on the membrane, loading viruses on the membrane.

20. A viral transduction and/or electroporation system, comprising:
a membrane;
electroporation electrodes; and
an electrical voltage source for establishing an electrical field at the membrane and between the electrodes,
means for tuning a distance between the electrodes and the membrane.

21. A viral transduction and/or electroporation system, comprising:
a membrane separating two cavities;
electroporation electrodes;
an electrical voltage source for establishing an electrical field at the membrane and between the electrodes;
an electroporation buffer reservoir for supplying electroporation buffer into the cavities;
a viral transduction reservoir for supplying viruses into the cavities; and
a cell media reservoir for supplying cell media into the cavities.

22. A viral transduction and/or electroporation system, comprising:
a membrane separating two cavities;
electroporation electrodes;
an electrical voltage source for establishing an electrical field at the membrane and between the electrodes;
a pinning pump for pinning particles against the membrane; and
an unpinning pump for unpinning particles from the membrane.
